(19) United States
(12) Patent Application Publication
(10) Pub. No.: US 2019/0159929 A1
(43) Pub. Date: May 30, 2019

(54) MOIST HEAT DELIVERY THERAPY DEVICE WITH A MULTI-LAYER LAMINATE STRUCTURE

(71) Applicant: BRUDER HEALTHCARE COMPANY, LLC, Alpharetta, GA (US)

(72) Inventor: Mark H. BRUDER, Alpharetta, GA (US)

(73) Assignee: BRUDER HEALTHCARE COMPANY, LLC, Alpharetta, GA (US)

(21) Appl. No.: 16/202,879

(22) Filed: Nov. 28, 2018

Related U.S. Application Data
(60) Provisional application No. 62/591,929, filed on Nov. 29, 2017.

(51) Int. Cl. A61F 7/02; A61F 9/04; A61D 9/00; A61F 7/00

(52) U.S. Cl. CPC A61F 7/02; A61F 9/04; A61D 9/00; A61F 7/0085; A61F 2007/0004; A61F 2007/0228; A61F 2007/0257; A61F 2007/0258; A61F 2007/0268; A61F 2007/0204; A61F 2007/0222; A61F 2007/0223; A61F 2007/0242

(57) ABSTRACT

A therapeutic device such as an eye mask for delivery of moist heat therapy to a body part such as an eye of a human or animal subject. Example devices are configured to be worn with one or more eye coverage portions positioned over the subject's eye(s). The eye coverage portions have a front side configured for placement against the treated body part, and a back side opposite the front side. A moisture barrier material positioned at or adjacent the back side substantially prevents moisture from escaping from the back side and directs moist heat delivery through the front side and toward the body part to be treated.

### CROSS-REFERENCE TO RELATED APPLICATION

This application claims the benefit of U.S. Provisional Patent Application Ser. No. 62/591,929 filed Nov. 29, 2017, the entirety of which is hereby incorporated herein by reference for all purposes.

### TECHNICAL FIELD

The present invention relates generally to the field of medical therapy by delivery of moist heat to a body part of a human or animal subject. In particular applications, embodiments of the invention relate to ophthalmic medical therapy or palliative care of the eye, and further relate to a moist heat delivery therapy device with a laminate or directive structure for directing moist heat toward the treated body part and preventing loss of moist heat from the therapy device away from the treated body part.

### BACKGROUND

Various conditions of the eye may require medical or palliative care. For example, blepharitis is a common and ongoing condition where the eyelids become inflamed (swollen) with oily particles and bacteria coating the eyelid margin near the base of the eyelashes. This condition causes irritation, itchiness, redness, dry eye and stinging or burning of the eyes. While the underlying causes of blepharitis are not completely understood, it can be associated with a bacterial eye infection, symptoms of dry eyes or certain types of skin conditions, such as acne rosacea. Anterior blepharitis affects the outside of the eyelid where the eyelashes are attached. This can be caused by bacterial (or sometimes viral) infection. If left untreated, anterior blepharitis can lead to thickened and inward-turned or outward-turned eyelids. Untreated anterior blepharitis can even lead to vision problems from in-turned eyelashes damaging the cornea. Posterior blepharitis is a condition that results from a dysfunction of the eye's tiny oil glands (meibomian glands) in the eyelids at the base of the eyelashes. When meibomian glands become clogged from posterior blepharitis, it can cause a stye or chalazion to form. Posterior blepharitis can also lead to thickened eyelid margins and crusty eyelids.

An estimated 40.9 million people in the United States aged 18 or older wear contact lenses. The International Workshop on Contact Lens Discomfort, published in 2013, put forth dryness of the eyes as a primary reason for contact lens intolerance. When a contact lens is place on the eye, the tear film structure becomes altered resulting in a pre-lens thinned lipid layer and a post-lens thinned aqueous layer. As a result of this disruption from the contact lens, the tear film tends to have an increased rate of evaporation leading to poor wetting on the surface of the contact lens and inadequate lubrication on the surface of the eye. This is further exacerbated if the patient has an already unstable lipid layer due to the presence of meibomian gland dysfunction (MGD). MGD is considered by many to be the leading cause of dry eye disease throughout the world, and is a chronic and progressive condition that can contribute to a poor quality lipid layer and lead to contact lens discomfort. Contact lens wearers often report dry eye symptoms and show signs of MGD, including gland atrophy, thinned lipid layer, and increased tear film instability. MGD has been observed in many patients with intolerance to contact lenses. Therefore, treatment of MGD may improve meibomian gland functions and relieve discomfort from dry eyes and contact lenses.

### SUMMARY

It has been found that thermal therapy can be used for medical or palliative care of a human or animal subject or patient, for example by delivering moist heat or cold therapy to the eye region. In example applications, thermal therapy can be used to unblock glands in the eye to help treat dry eye. Moist heat therapy may also be used to help reduce elevated intraocular pressure to either treat or help prevent open-angle glaucoma. Delivery of medications to the eyes, such as for treatment of blepharitis may be enhanced by application of thermal therapy in combination with the medication. Applying heat to the inner eyelid may also help safely remove gland obstructions and stagnant gland content.

In example embodiments, the present invention provides a therapeutic eye mask or compress generally including at least one, and in particular embodiments two separate eye coverage portions—each configured to deliver moist heat therapy. The mask is configured to be worn on the head of a human or animal patient with one of the eye coverage portions positioned over at least one eye, and in particular embodiments over both eyes of the patient. Each eye coverage portion has a front side, configured to rest against the face of the patient, and a back side opposite the front side. In example embodiments, each eye coverage portion includes a moisture barrier material—positioned towards the back side of the eye coverage portion—configured to prevent moisture from escaping from the back side of the eye coverage portion, thereby directing delivery of moist heat therapy toward the patient.

In one aspect, the present invention relates to an eye mask configured to deliver moist heat therapy to at least one eye of a patient—the eye mask comprising at least one eye coverage portion and at least one securing strap. Further the at least one eye coverage portion comprises a fill material configured to absorb and release moisture, such as water; a moisture-permeable material surrounding and containing the fill material; and a moisture-impermeable material attached to a surface of the moisture permeable material. The moisture-impermeable material is configured to at least partially contain and/or direct moisture released from the fill material.

In another aspect, the invention relates to an eye compress for delivery of moist heat to a patient. In example forms, the eye compress includes at least one enclosure containing a fill material capable of absorbing and releasing moist heat for therapeutic delivery to the patient, the enclosure having a first side and an opposite second side, with the fill material disposed between the first side and the second side, and wherein the first side is configured for application to a body portion of the patient. In example embodiments, the eye compress also includes a moisture barrier positioned along the second side, whereby moist heat is delivered from the fill material in a first direction toward and through the first side, and whereby moist heat is substantially prevented from transmission from the fill material in a second direction toward the second side.

In still another aspect, the invention relates to a method of providing moist heat therapy to a body part of a human or animal subject. In example forms, the method includes providing a therapeutic device comprising a first side, a second side, and a hydrophilic fill material contained between the first and second sides; wherein the first side at least partially comprises a moisture-transmissive material allowing passage of moisture therethrough for absorption and release into and from the fill material, and the second side at least partially comprises a moisture-impermeable material substantially preventing passage of moisture therethrough.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of example embodiments are explanatory of example embodiments of the invention, and are not restrictive of the invention, as claimed.

### BRIEF DESCRIPTION OF THE DRAWINGS

FIG. 1 is a perspective view of a therapeutic eye mask according to an example embodiment of the present invention.

FIG. 2 shows the therapeutic eye mask of FIG. 1 worn by a human patient.

FIG. 3 is a detailed view of the back side of an eye coverage portion of the therapeutic eye mask of FIG. 1.

FIG. 4 is a cutaway view of the eye coverage portion of FIG. 3, wherein the outer cover layer is retracted.

FIG. 5 is a cutaway view of the eye coverage portion of FIG. 3, wherein the outer cover layer and intermediate layers are retracted.

FIG. 6 is a cutaway view of the eye coverage portion of FIG. 3, wherein the outer cover layer, intermediate layer, the moisture impermeable panel, and the inner pouch are retracted.

FIG. 7 is a partial cross-section of an eye coverage portion of the therapeutic eye mask of FIG. 1.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

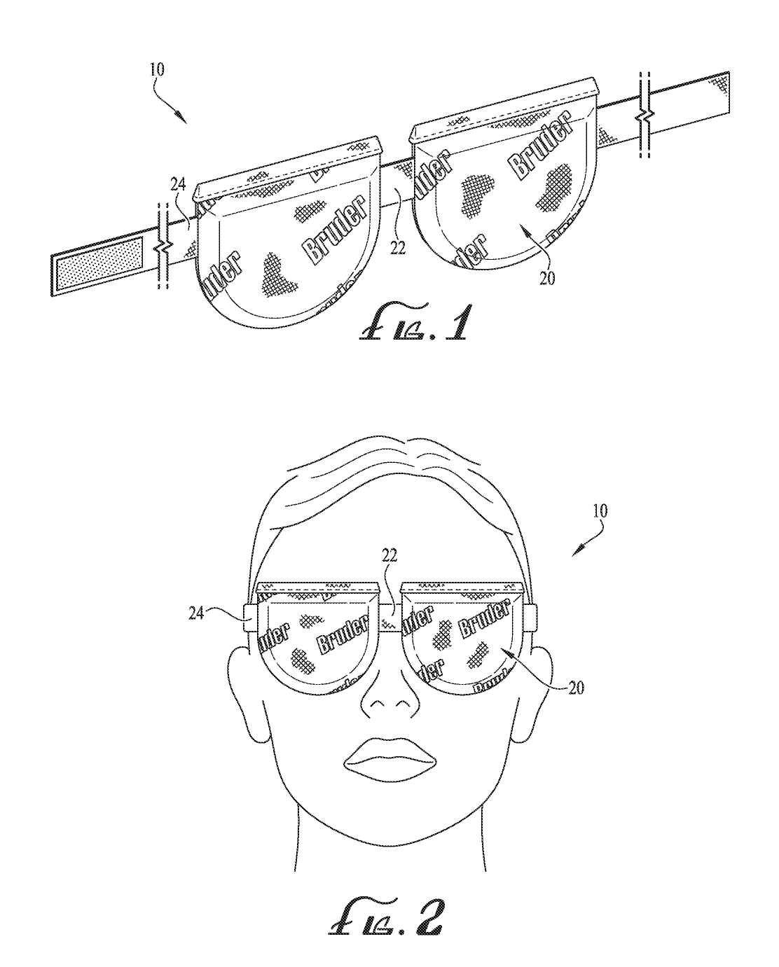

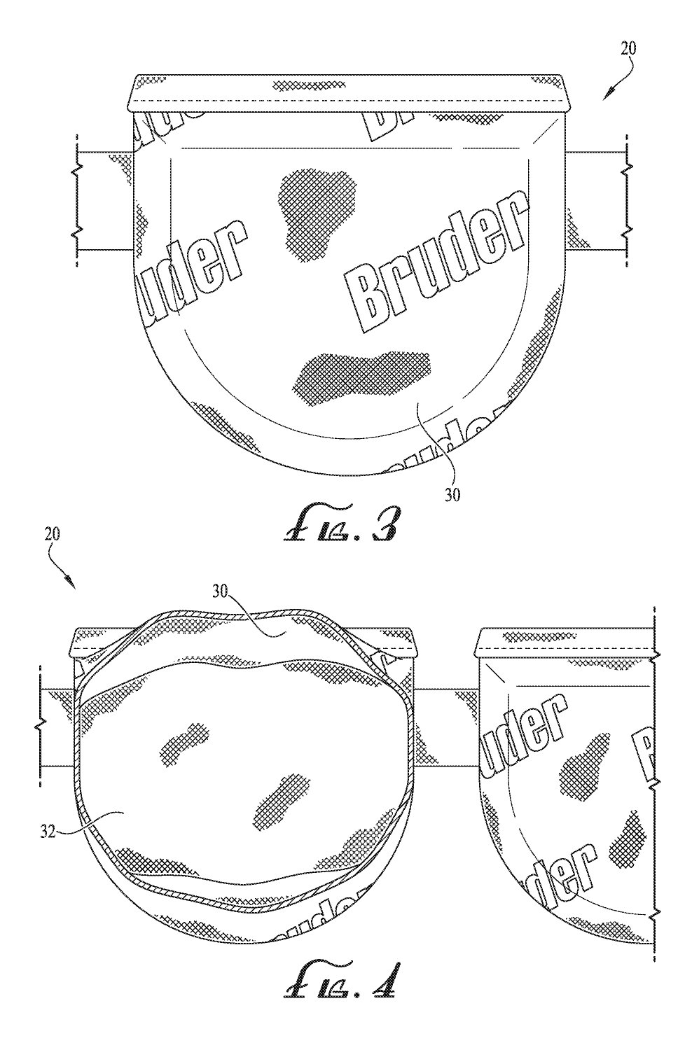

With reference now to the drawing figures, wherein like reference numbers represent corresponding parts throughout the several views, FIGS. 1-7 show a therapeutic eye mask or compress according to an example embodiment of the present invention. The therapeutic eye mask 10 generally includes at least one, and in the depicted embodiment two separate eye coverage portions 20, each configured to deliver moist heat therapy, connected by a nose bridge 22 and a securing strap 24, as shown in FIG. 1. In example embodiments, the nose bridge 22 and/or the securing strap 24 may comprise a flexible elastic material or strip, and optionally also comprise one or more attachment or connection elements such as hook-and-loop attachment material, snaps, clips, buttons or the like, to allow attachment and optionally adjustment of the fit around the user's head. The mask 10 is configured to be worn on the head of a human or animal patient with one of the eye coverage portions 20 positioned over each eye of the patient. In example embodiments, the securing strap 24 is positioned around the user's head to hold the eye coverage portions 20 against or adjacent over the eyes of the user, as shown in FIG. 2. In alternate embodiments, a mask or compress having a single eye coverage portion is provided, allowing the user to treat one eye at a time, leaving the other eye uncovered. Each eye coverage portion 20 has a front side or end 26, configured to rest against the face of the patient (the inner or patient treatment side of the compress), and a back side or end 28 opposite the front side or end (the outer or distal side of the compress). FIG. 3 shows a detailed view of the back side 28 of an eye coverage portion 20. In example embodiments, the inner or front side 26 of the eye coverage portion 20 has a different appearance and/or texture than the outer or back side 28, to allow a user to readily differentiate which side of the compress is the patient treatment side to be applied over the patient's eye region. For example, the inner or front side may comprise a smoother or softer material without coloration or printing, whereas the outer or back side may comprise a more durable or rougher textured material having branding text or logos, or other coloration or indicia imprinted or otherwise applied thereon. Alternatively, instructional information may be imprinted or otherwise applied on one or both sides (for example, “this side toward you” printed on the inner or front side). Each eye coverage portion 20 includes a moisture barrier material 34, positioned towards the back or outer side 28 of the eye coverage portion, configured to prevent moisture from escaping from the back or outer side of the eye coverage portion, thereby directing moist heat therapy toward the front or inner side for application to the eye region of the patient (i.e., in a patient-treatment direction).

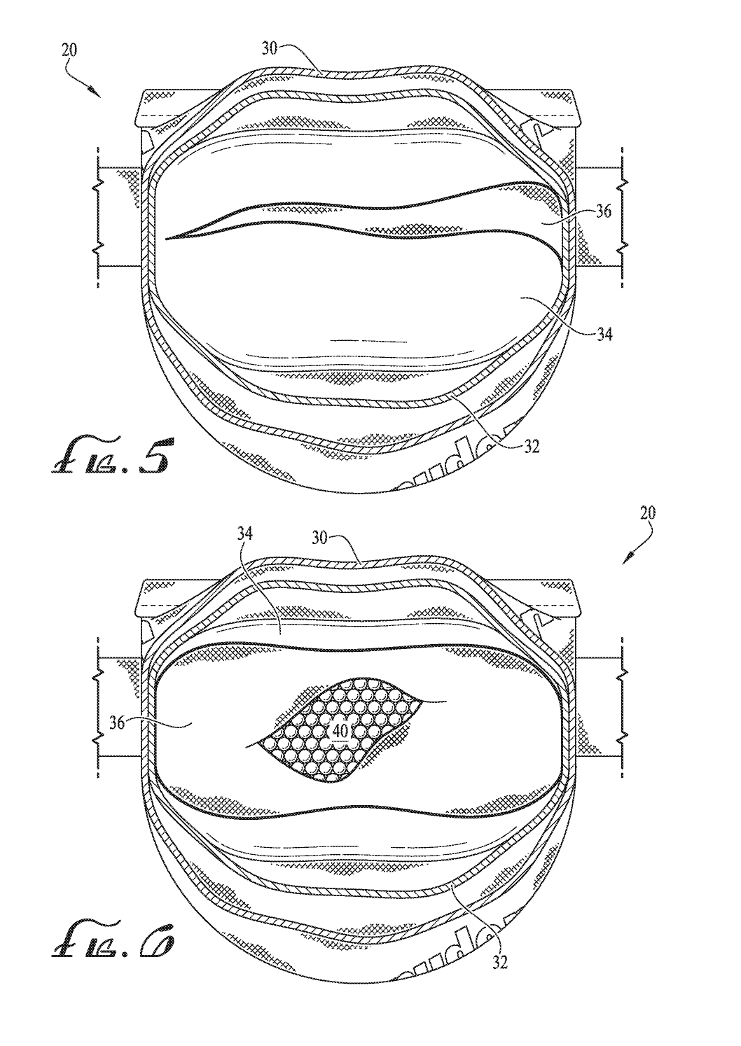

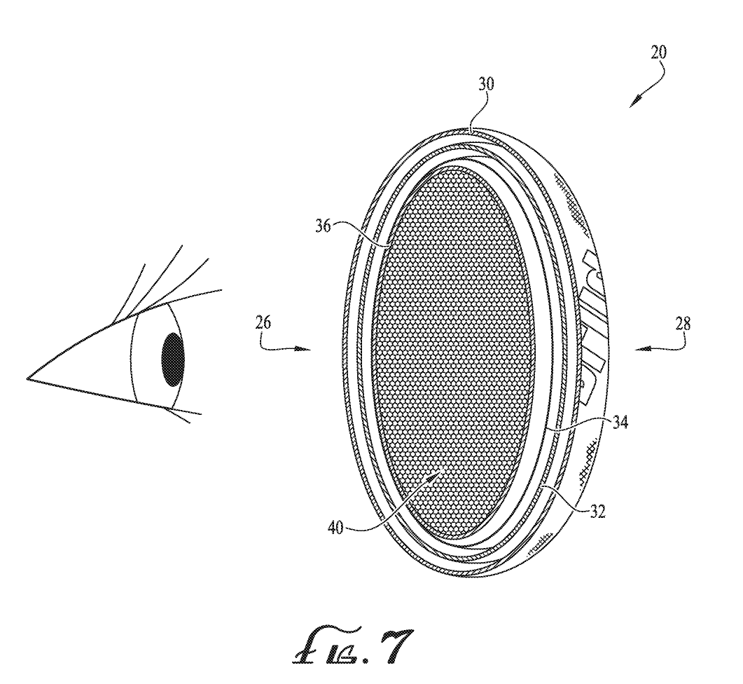

In example embodiments, the eye coverage portions 20 are independent structures such that each can independently conform to the respective eye region of the patient. For example, in the depicted embodiment as shown in greater detail in FIG. 3, the eye coverage portion 20 has a radially curved lower or bottom profile, and a generally rectangular upper or top profile, defining a generally U-shaped configuration. Optionally, an upper attachment strip is stitched along the top edge of the eye coverage portion. Alternatively, the eye coverage portions can be combined in a single panel or mask format configured to extend over both eyes. In the depicted embodiment, each eye cover portion 20 is formed from a series of layers or lamina. FIGS. 4-6 show successive layers being cut away and retracted to show the series of layers or lamina, and FIG. 7 shows a cross-sectioned view of a portion of an eye cover with the successive layers or lamina indicated. For example, the eye coverage portion 20 can include an outer cover 30 configured to surround the eye coverage portion 20. The outer cover 30 generally surrounds both the front side 26 and back side 28 of the eye coverage portion 20, as shown in FIG. 7. The eye coverage portion also includes an inner pouch 36 that holds a fill material 40, as shown in FIG. 6, configured to retain moisture and deliver moist heat. The outer cover 30 is configured to surround and contain the inner pouch 36. The inner pouch 36 and outer cover 30 are preferably at least partially moisture permeable such that moisture and heat (moist heat) can travel from the fill material 40 to the face of the patient. In example embodiments, the eye coverage portion 20 also includes a panel or sheet 34 of moisture impermeable or minimally-moisture permeable material. The panel 34 is positioned between the inner pouch 36 and the outer cover 30 of the eye coverage portion 20. The panel 34 is generally positioned towards the back or outer side 28 of the eye coverage portion 20 such that the panel is not positioned between the inner pouch 36 and the face of the patient, as shown in FIG. 7. The moisture impermeable panel 34 is configured to help prevent the moisture released from the fill material 40 from escaping through the back end 28 of the eye coverage portion 20, and better direct moist heat to the subject for treatment. In example embodiments, the panel 34 can be simply held between the outer cover 30 and the inner pouch 36. In other embodiments, the panel 34 can be attached to the outer cover 30, the inner pouch 36, or another part of the eye coverage portion. Example attachment means include gluing, sewing, or other fastening means. In other embodiments, the moisture barrier panel 34 is laminated to another layer of the eye coverage portion to form a unitary structure. The panel 34 can be flame laminated to eliminate potential for skin contact with any irritant adhesive materials. For example, the panel 34 can be laminated to the outer rear surface of the inner pouch 36 or the inner rear surface of the outer cover 30.

In other embodiments, the eye coverage portions 20 can include an intermediate layer 32 in between the inner pouch 36 and the outer cover 30, as shown in FIGS. 4 and 5. The intermediate layer 32 is generally formed from a moisture permeable material. In example embodiments, the intermediate layer 32 surrounds and contains the inner pouch 36. In other embodiments, the intermediate layer 32 covers only a portion of the inner pouch 36. In these embodiments, the moisture-barrier panel 34 can be attached or laminated to the inner rear surface or the outer rear surface of the intermediate layer 32.

In example embodiments, the outer cover 30, inner pouch 36, and intermediate layer 32 are constructed of a lightweight, durable, and flexible material such as foam or polyester. The material is configured to allow moist heat to reach the face of the patient, but limit the amount of moisture released from the fill material. In example embodiments, one or more of the layers 30/32/36 are formed from an open cell thermoplastic polyurethane foam that is about 2 mm thick. In other examples, one or more of the layers 30/32/36 are constructed from a 2 lb/ft³ (pounds per cubic foot), ⅜″ thick polyether polyurethane foam. The foam layers can include polyester fabric laminated onto one side. Alternatively, one or more of the layers 30/32/36 are constructed from polyester, rayon, spandex, silk, polyethylene, neoprene, ECA, ethylene-vinyl acetate (EVA), other plastic films, and/or other natural and/or synthetic fabrics or materials having the same or substantially similar characteristics and capabilities. For example, one or more of the layers 30/32/36 can be formed from polyester felt. In other embodiments, one or more of the layers 30/32/36 are formed from a closed cell foam. The foam can be polyurethane and/or thermoformable. The outer cover 30 can alternatively be constructed from a woven material different from the inner pouch 36 and intermediate layer 32. The mask material can optionally be selected to have insulative or heat-transmissive properties to affect the temperature transferred from the mask to the patient's eyes, better ensuring safety. The material may optionally be washable for reuse, or alternatively can be a single-use disposable product. In example embodiments, the eye coverage portions 20 can be constructed of a material containing nanobeads comprising an antimicrobial metal, medications, and/or other therapeutic material(s). Optionally, the mask 10 may be configured for use in connection with medicated or therapeutic sheets, pods or other therapeutic accessories positioned or retained between the eye coverage portions 20 and the subject's eye areas. For example, the mask 10 may be configured for supplemental delivery and transport of medication or therapeutic material with moist heat.

The moisture barrier panel or material 34 can be constructed of a flexible material that will limit moisture that may otherwise be lost through the open cell foam, e.g., a plastic material. In example embodiments, the panel 34 is formed of a multilayer structure of nylon, ethylene vinyl alcohol (EVOH), and polyethylene (PE) that is about 4 mm thick, other plastic films, and/or other natural and/or synthetic fabrics or materials having the same or substantially similar characteristics and capabilities.

In example embodiments, the fill material 40 is loosely contained within the inner pouch 36 and comprises a synthetic porous crystalline granular aluminosilicate zeolite—e.g., a hydrophilic natural or synthetic zeolite, also referred to as a molecular sieve material—or other substances with similar properties. In the depicted embodiment, the fill material 40 comprises a multiplicity of individual solid beads or granules that absorb and release moisture while generally maintaining their individual solid shape and consistency. The fill material 40 is surrounded and contained by the inner pouch 36 such that the fill material is unable to escape from the inner pouch 36. The fill material 40 may further comprise other inert additives and physical matrices without affecting the antimicrobial and hydrous efficacies of the fill. The hydrophilic zeolite granules or beads are configured to repeatedly absorb and release moisture without substantially changing shape or form. Optionally, the fill material 40 comprise a granular material such as activated alumina, silica gel, bentonite or hydrophilic zeolite or molecular sieve material loosely contained in the pouch or other enclosure. In alternate embodiments, the fill material comprises capsules or packets of non-granular material (e.g., gel, liquid), powder, or other materials. The fill material 40 optionally also contains a metallic or other antimicrobial agent, such as for example silver, copper, copper oxide, gold, magnesium oxide, aluminum oxide, titanium dioxide, zinc oxide, cobalt, nickel, zirconium, molybdenum, tin, lead and/or other metals; metal oxides, metal ions, metal particles or nanoparticles; and alloys, mixtures or combinations thereof deposited therein. For example, silver or other metal loading of the fill material 40 may be attained by the process of ion-exchange. In this process, a solution containing atomic silver, or a composition of silver, bathes or is passed through a bed of the fill granules. An ion-exchange column method may be performed in which an aqueous solution containing atomic silver or a composition of silver may be passed through a column bed of the fill granules, and the eluted solution may again be passed through the bed or may receive additional silver and then be again passed through the bed.

Various ion-exchange schedules as known in the art may be applied to produce retention of the silver or other metals in the fill material 40. For example, the final content by weight of an atomic silver or silver composition, or other metals or antimicrobial agents, may be as high as twenty percent of the final loaded fill granules. In example embodiments, the loaded fill granules produced by ion-exchange will exhibit high retention of the silver or other metals even under subsequent exposure to fluids and microwave irradiation. The fill granules may comprise a blend of both metal loaded and unloaded (i.e., not containing metal) zeolite or other substance(s) retaining silver or other metals. The presence of the atomic silver or other metals preferably will not interfere with the useful properties of the fill granules such as the moisture desorption, absorption, and/or adsorption properties which may be desirable in the use of the eye mask or compress system. The hydrophilic nature of example forms of zeolite fill materials provides that substantial water content is available therein and readily replenished by absorption of moisture in the form of water vapor from the atmosphere or ambient surroundings at standard room temperatures and conditions. The water so absorbed may be sufficient for moist heat delivery applications, or may be supplemented by manually added water, for providing a microwave responsive water content of the eye mask or compress system. The compositions of silver or other metals used may include but are not limited to, metal compounds, and metal salts such as silver chloride and silver nitrate.

The presence of silver or other metals within the fill granules, while optional, may provide anti-microbial properties to the therapeutic eye mask system. The ion-exchange loaded fill granules will preferably retain the silver or other metals despite microwave heating as may be required in the use of the eye mask or compress system. Further, the retention of the silver or other metals within the fill granules provides assured antimicrobial performance in a reusable and potentially washable, if so desired, moist heat therapy compress. In other embodiments, silver or other metals are incorporated into the cover material and/or other portions of the eye mask system, in addition to or instead of the fill granules. Alternatively, one or more non-metal antimicrobial materials and/or medications may optionally be incorporated into the fill material 40, the eye coverage portions 20, and/or other portions of the eye mask system.

The present invention also includes a method of providing moist heat therapy to a body part of a human or animal subject. In example forms, the method includes providing a therapeutic device comprising a first side, a second side, and a hydrophilic fill material contained between the first and second sides; wherein the first side at least partially comprises a moisture-transmissive material allowing passage of moisture therethrough for absorption and release into and from the fill material, and the second side at least partially comprises a moisture-impermeable material substantially preventing passage of moisture therethrough. In further example embodiments, the body part of the human or animal subject is an eye. In further example embodiments, the method also includes exposing the therapeutic device to a moisture source, such as for example, water vapor in ambient air surrounding the therapeutic device to allow absorption of moisture into the hydrophilic fill material. In further example embodiments, the method further includes exposing the therapeutic device to a heat source, such as for example, a microwave oven or other heating device to allow transfer of heat into the fill material. In further example embodiments, the method further includes directing the application of the first side of the therapeutic device into contact with the body part to provide transmission of moist heat in a first direction from the hydrophilic fill material through the moisture-transmissive material to the body part, and to substantially prevent transmission of moist heat in an opposed second direction from the hydrophilic fill material through the moisture-impermeable material away from the body part. In further example embodiments, the method further includes applying the first side of the therapeutic device into contact with the body part to provide transmission of moist heat in a first direction from the hydrophilic fill material through the moisture-transmissive material to the body part, and to substantially prevent transmission of moist heat in an opposed second direction from the hydrophilic fill material through the moisture-impermeable material away from the body part.

While the invention has been described with reference to example embodiments, it will be understood by those skilled in the art that a variety of modifications, additions and deletions are within the scope of the invention, as defined by the following claims.

What is claimed is:

1. An eye mask configured to deliver moist heat therapy to at least one eye of a patient, the eye mask comprising:
at least one eye coverage portion; and
at least one securing strap affixed to the eye coverage portion;
wherein the at least one eye coverage portion comprises an enclosure containing a fill material configured to absorb and release moisture, a moisture-permeable material at least partially surrounding and containing the fill material, and a moisture-impermeable material configured to at least partially prevent moisture transmission from the fill material in a first direction, whereby moisture from the fill material is substantially directed toward the patient in a second direction generally opposite the first direction.

2. The eye mask of claim 1, comprising two independent eye coverage portions and a flexible nose bridge coupled between the two eye coverage portions.

3. The eye mask of claim 2, wherein the flexible nose bridge is adjustable.

4. The eye mask of claim 1, wherein the fill material comprises a hydrophilic zeolite.

5. The eye mask of claim 4, wherein the hydrophilic zeolite fill material comprises granules loosely contained within the enclosure.

6. The eye mask of claim 4, wherein the fill material contains an antimicrobial material.

7. The eye mask of claim 1, wherein the moisture-impermeable material is laminated to the moisture-permeable material.

8. The eye mask of claim 1, wherein the moisture-impermeable material is attached to the back side of the moisture permeable material.

9. The eye mask of claim 1, wherein the moisture-permeable material surrounding and containing the fill material is surrounded and contained by a second moisture-permeable material.

10. The eye mask of claim 1, wherein the moisture-permeable material is selected from an open cell thermoplastic polyurethane foam, a polyether polyurethane foam, a polyester fabric, rayon, spandex, silk, polyethylene, neoprene, ECA, ethylene-vinyl acetate (EVA), polyester felt or other natural and/or synthetic fabrics or materials, and combinations thereof.

11. The eye mask of claim 1, wherein the moisture-impermeable material is selected from a plastic material, nylon, EVOH, or PE, or other natural and/or synthetic fabrics or materials, and combinations thereof.

12. An eye compress for delivery of moist heat to a patient, the eye compress comprising:
at least one enclosure containing a fill material capable of absorbing and releasing moist heat for therapeutic delivery to the patient, the enclosure having a first side and an opposite second side, with the fill material disposed between the first side and the second side, and wherein the first side is configured for application to a body portion of the patient; and
a moisture barrier positioned along the second side, whereby moist heat is delivered from the fill material in a first direction toward and through the first side, and whereby moist heat is substantially prevented from transmission from the fill material in a second direction toward the second side.

13. The eye compress of claim 12, comprising a pair of enclosures containing the fill material, each of the pair of enclosures configured for application over a respective one of the patient's eyes.

14. The eye compress of claim 12, further comprising a nose bridge portion coupled between the pair of enclosures.

15. The eye compress of claim 12, further comprising a head strap for retaining the eye compress over an eye region of the patient.

16. The eye compress of claim 12, wherein the fill material comprises a hydrophilic zeolite.

17. The eye mask of claim 16, wherein the hydrophilic zeolite fill material comprises granules loosely contained within the enclosure.

18. The eye mask of claim 16, wherein the fill material contains an antimicrobial material.

19. The eye compress of claim 12, wherein the moisture barrier comprises a material selected from a moisture-impermeable plastic material, nylon, EVOH, or PE, or other natural and/or synthetic moisture-impermeable fabrics or materials, and combinations thereof.

20. A method of providing moist heat therapy to a body part of a human or animal subject, the method comprising:
providing a therapeutic device comprising a first side, a second side, and a hydrophilic fill material contained between the first and second sides, the first side at least partially comprising a moisture-transmissive material allowing passage of moisture therethrough for absorption and release into and from the fill material, and the second side at least partially comprising a moisture-impermeable material substantially preventing passage of moisture therethrough.

21. The method of claim 20, wherein the body part of the human or animal subject is an eye.

22. The method of claim 20, further comprising exposing the therapeutic device to a moisture source to allow absorption of moisture into the hydrophilic fill material.

23. The method of claim 22, wherein the moisture source is water vapor in ambient air surrounding the therapeutic device.

24. The method of claim 20, further comprising exposing the therapeutic device to a heat source to allow transfer of heat into the fill material.

23. The method of claim 24, wherein the heat source is a microwave heating device.

24. The method of claim 20, further comprising directing the application of the first side of the therapeutic device into contact with the body part to provide transmission of moist heat in a first direction from the hydrophilic fill material through the moisture-transmissive material to the body part, and to substantially prevent transmission of moist heat in an opposed second direction from the hydrophilic fill material through the moisture-impermeable material away from the body part.

25. The method of claim 20, further comprising applying the first side of the therapeutic device into contact with the body part to provide transmission of moist heat in a first direction from the hydrophilic fill material through the moisture-transmissive material to the body part, and to substantially prevent transmission of moist heat in an opposed second direction from the hydrophilic fill material through the moisture-impermeable material away from the body part.
